(19) United States
(12) Patent Application Publication
(10) Pub. No.: US 2015/0073294 A1
(43) Pub. Date: Mar. 12, 2015

(54) METHOD FOR ASSESSING THE TREATMENT OF ATTENTION-DEFICIT/HYPERACTIVITY DISORDER

(71) Applicants: AGENCY FOR SCIENCE, TECHNOLOGY RESEARCH, Singapore (SG); NATIONAL UNIVERSITY OF SINGAPORE, Singapore (SG); INSTITUTE OF MENTAL HEALTH, Singapore (SG)

(72) Inventors: Haihong Zhang, Singapore (SG); Cuntai Guan, Singapore (SG); Ranga Krishnan, Singapore (SG); Tih-Shih Lee, Singapore (SG); Choon Guan Lim, Singapore (SG); Daniel Fung, Singapore (SG)

(21) Appl. No.: 14/389,751

(22) PCT Filed: Mar. 28, 2013
(86) PCT No.: PCT/SG2013/000122

§ 371 Date: Sep. 30, 2014

Related U.S. Application Data
(60) Provisional application No. 61/618,618, filed on Mar. 30, 2012.

(51) Int. Cl. A61B 5/16; A61B 5/00; G06K 9/00; A61B 5/0476

(52) U.S. Cl. CPC A61B 5/168; A61B 5/0476; A61B 5/7275; G06K 9/00523; G06K 9/00536
USPC 600/544

(57) ABSTRACT

According to one aspect, there is provided a method for assessing the treatment of attention-deficit/hyperactivity disorder (ADHD) in a subject, the method comprising: obtaining electroencephalographic (EEG) data relating to a plurality of subjects diagnosed with ADHD; extracting, for each of the plurality of subjects, at least one feature from the EEG data relating to that subject; formulating a prediction model by performing regression analysis to map the extracted features against one or more markers for each of the plurality of subjects; and determining that the prediction model provides an ADHD assessment if one or more of the markers are indicators of a clinical measure of interest.

### FIELD OF INVENTION

The invention relates generally to a method for assessing the treatment of attention-deficit/hyperactivity disorder (ADHD).

### BACKGROUND

Attention-deficit/hyperactivity disorder (ADHD) is a common psychiatric condition and a neuro developmental disorder with various hyperactivity, impulsivity and inattention symptoms. Stimulant medication is widely used to treat ADHD, but there are concerns like medication adverse effects, as well as long-term efficacy. Therefore, alternative interventions are called for to treat non-responders or a large percentage of patients who do not accept/sustain stimulant medication.

Electroencephalographic (EEG) biofeedback (a.k.a neurofeedback) is one such alternative treatment. It is an operant conditioning procedure in which the patient learns to self-control certain EEG patterns, guided by real-time visual or acoustic representations of the EEG patterns.

There are basically two major neurofeedback approaches, self-regulation of band powers and/or their ratios, and that of slow-cortical potentials, which are motivated by two groups of findings of EEG characteristics in children with ADHD, one, the increased Theta and decreased Alpha and Beta powers; and two, the differences in event-related potentials.

Another treatment method explores beyond the neurofeedback approach. Specifically, individual subjects' attention conditions are decoded from EEG using a subject-specific attention detection model, and associated directly with the control signal in therapeutic video games.

With existing techniques and tools, diagnosis is made by assessing symptoms and testing attention, and the severity is assessed based upon severity of symptoms. Children mature at different rates and have different personalities, temperaments, and energy levels. Most children get distracted, act impulsively, and struggle to concentrate at one time or another. As symptoms vary from person to person, the disorder can be hard to diagnose. For example, normal behaviours may be mistaken for ADHD. As there is no single test can diagnose a child as having ADHD, a licensed health professional needs to gather information about the child, and his or her behavior and environment. In addition, treatment severity is used as the basis for adjusting treatment including dose of medication. This is difficult to obtain in a reliable manner and involves collecting data from family, school and others.

A need therefore exists to provide an objective assessment of ADHD severity, which can be used to predict ADHD treatment response.

### SUMMARY

According to a first aspect, there is provided a method for assessing the treatment of attention-deficit/hyperactivity disorder (ADHD) in a subject, the method comprising: obtaining electroencephalographic (EEG) data relating to a plurality of subjects diagnosed with ADHD; extracting, for each of the plurality of subjects, at least one feature from the EEG data relating to that subject; formulating a prediction model by performing regression analysis to map the extracted features against one or more markers for each of the plurality of subjects; and determining that the prediction model provides an ADHD assessment if one or more of the markers are indicators of a clinical measure of interest.

According to a second aspect, there is provided an apparatus for assessing the treatment of attention-deficit/hyperactivity disorder (ADHD) in a subject, the apparatus comprising: at least one processor; and at least one memory including computer program code, the at least one memory and the computer program code configured to, with the at least one processor, cause the apparatus at least to perform: obtaining electroencephalographic (EEG) data relating to a plurality of subjects diagnosed with ADHD; extracting, for each of the plurality of subjects, at least one feature from the EEG data relating to that subject; formulating a prediction model by performing regression analysis to map the extracted features against one or more markers for each of the plurality of subjects; and determining that the prediction model provides an ADHD assessment if one or more of the markers are indicators of a clinical measure of interest.

According to a third aspect, there is provided a computer readable medium for assessing the treatment of attention-deficit/hyperactivity disorder (ADHD) in a subject, the computer readable medium having stored thereon computer program code which when executed by a computer causes the computer to perform at least the following: obtaining electroencephalographic (EEG) data relating to a plurality of subjects diagnosed with ADHD; extracting, for each of the plurality of subjects, at least one feature from the EEG data relating to that subject; formulating a prediction model by performing regression analysis to map the extracted features against one or more markers for each of the plurality of subjects; and determining that the prediction model provides an ADHD assessment if one or more of the markers are indicators of a clinical measure of interest.

### BRIEF DESCRIPTION OF THE DRAWINGS

Example embodiments of the invention will be better understood and readily apparent to one of ordinary skill in the art from the following written description, by way of example only, and in conjunction with the drawings. The drawings are not necessarily to scale, emphasis instead generally being placed upon illustrating the principles of the invention, in which:

FIG. 1 shows a flowchart that illustrates a method for assessing the treatment of attention-deficit/hyperactivity disorder (ADHD) according to a first embodiment.

FIG. 2 shows an apparatus, according to a second embodiment, for assessing the treatment of attention-deficit/hyperactivity disorder (ADHD) in a subject.

FIGS. 3 and 4 are plots of the correlation between change in ADHD rating scores from Week 0 to Week 20 for ADHD inattention and hyperactivity symptoms, and a combination of both.

FIG. 5 illustrates an exemplary application for the above embodiments.

FIG. 6 shows a picture of a setup for brain computer interface training.

FIG. 7 is a graphical representation of the change in mean and standard error of ADHD-RS scores (ADHD rating score, 4^{th} edition).

### DEFINITIONS

The following provides sample, but not exhaustive, definitions for expressions used throughout various embodiments disclosed herein.

The phrase “clinical measure of interest” may mean parameters found in known rating scores, such as ADHD IA (ADHD—Predominantly Inattentive Type) rating scores based on DSM IV (Diagnostic and Statistical Manual of Mental Disorders-Fourth Edition).

### DETAILED DESCRIPTION

In the following description, various embodiments are described with reference to the drawings, where like reference characters generally refer to the same parts throughout the different views.

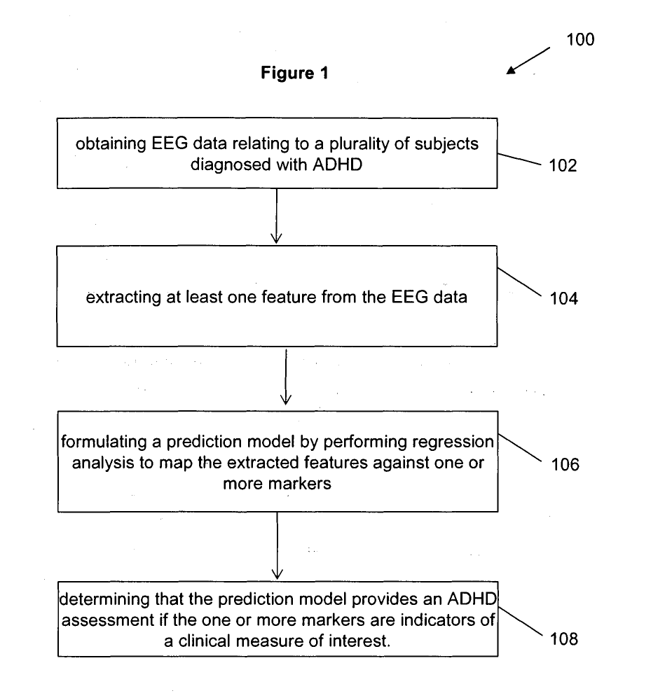

FIG. 1 shows a flowchart 100 that illustrates a method for assessing the treatment of attention-deficit/hyperactivity disorder (ADHD) according to a first embodiment. This methodology enables accurate clinical assessment using EEG only or with EEG plus clinical data and facilitates planning and tracking of progress of BCI-based (brain computer interface) treatment.

In step 102, electroencephalographic (EEG) data relating to a plurality of subjects diagnosed with ADHD is obtained. In step 104, for each of the plurality of subjects, at least one feature from the EEG data relating to that subject is extracted. In step 106, regression analysis to map the extracted features against one or more markers for each of the plurality of subjects is performed to formulate a prediction model. In step 108, it is determined that the prediction model provides an ADHD assessment if one or more of the markers are indicators of a clinical measure of interest.

In step 104, where features are extracted from EEG data, this facilitates filtering of data that does not assist in assessing ADHD, such as data relating to non-rhythmic patterns. The regression analysis that is conducted in step 106 is thus expedited. Regression is used for associating the features with an ADHD severity score. The regression analysis provides a means to design and validate a regression model that maps the extracted features to an assessment of ADHD. In one embodiment, regression may be casted as a tool for extrapolating or interpolating associations between variables. The results from step 108 provide for a score that may facilitate an objective assessment of ADHD severity, based on documented parameters or guidelines.

The EEG data may be from EEG signals measured by an EEG device, i.e. newly acquired data from an ADHD treatment session of the plurality of subjects diagnosed with ADHD. Clinically obtained ADHD scores (such as clinical assessment data that represents the clinical rating of ADHD severity) relating to the plurality of subjects may also be retrieved and used as input to the regression analysis to formulate the prediction model. The EEG data may be historical EEG data from a plurality of subjects, such as those recorded in previous ADHD treatment sessions or the previous medical history of the plurality of subjects. Accordingly, the EEG data may comprise historical EEG data relating to the plurality of subjects and/or historical clinical data relating to the plurality of subjects.

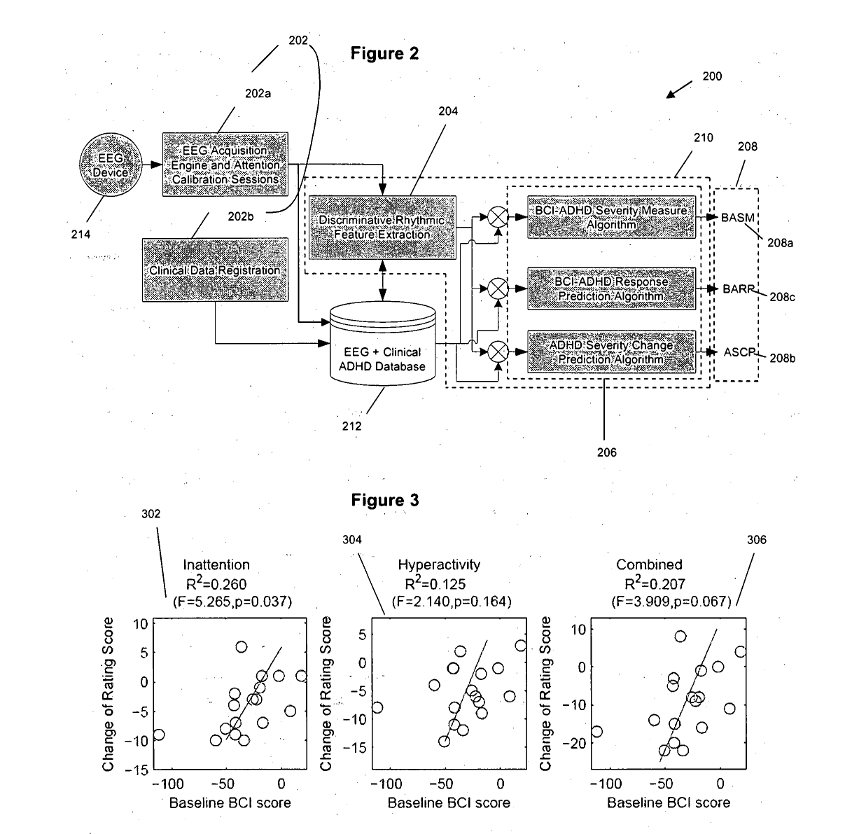

FIG. 2 shows an apparatus 200, according to a second embodiment, for assessing the treatment of attention-deficit/hyperactivity disorder (ADHD) in a subject.

The apparatus of FIG. 2 provides for a BCI (brain computer interface)-based methodology for ADHD assessment and prediction. EEG data and clinical data (such as clinically obtained ADHD scores relating to a plurality of subjects) is acquired (denoted using reference numeral 202), learned and key features extracted (denoted using reference numeral 204) for the purpose of prediction.

EEG data may be acquired from specific calibration sessions (denoted using reference numeral 202a), for example, sessions of interleaved Stroop tests and resting times. Clinical input is also acquired, for example from a clinical data registration database 202b. This clinical input may be clinical assessment data that represents the clinical rating of the ADHD severity. The clinical data and EEG data are stored and managed by a dedicated database, provided in memory 212.

Discriminative rhythmic features (DRFs) are extracted from the acquired EEG data using a complex-valued spatial-spectral filtering (CSSF) technique (denoted using reference numeral 204). Aspects of this CSSF will be elaborated in the following paragraphs.

DRFs and, if needed, corresponding clinical data, together with the history of one or both of the EEG data from which the DRFs are obtained and the clinical data, serve as the inputs to three measurement or prediction algorithms or prediction models that produce three scores:

1. BCI-based ADHD severity measure (BASM), that represents the severity of ADHD at the time of EEG recording and is also associated with a given specific clinical measure of interest, for example, ADHD IA rating scores based on DSM IV. This provides an ADHD severity score 208a at an instance of obtaining the EEG data for one subject;

2. BCI-ADHD Response Predictor (BARP), or ADHD predictor score 208c, that represents the estimated improvement of ADHD condition in terms of BASM from the time of EEG recording to a specified (pre-determined) time in future. This provides an ADHD assessment score for two time points, the first being an instance of obtaining the EEG data for one subject and the second being another instance where the EEG data has yet to be obtained; and

3. ADHD Severity Change Predictor (ASCP), or ADHD response score 208b, that represents the estimated improvement of ADHD condition in terms of BASM in between two EEG recording times. This provides an ADHD assessment score at two time points, being two instances of obtaining the EEG data for one subject.

Each of the three above scores, being a category of ADHD assessment, is the output of a respective prediction model for a particular clinical measure of interest, whereby the respective prediction model is formulated by regression analysis mapping extracted features that are indicative of that particular clinical measure of interest. Accordingly, a prediction model that is formulated depends on the extracted features that are mapped, so that the formulated prediction model provides a different ADHD assessment score. Each respective prediction model that provides one of the above three scores is, in one embodiment, independently derived. On the other hand, in another embodiment, the prediction model for the ADHD response score and/or for the ADHD predictor score may be based on the prediction model for the ADHD severity score.

Thus, in one embodiment, a different prediction model may be used to obtain each category of ADHD assessment. In another embodiment, the prediction model used to obtain a first of the ADHD assessment (e.g. the ADHD response score or for the ADHD predictor score) is based on the prediction model used to obtain a second of the ADHD assessment (e.g. the ADHD severity score).

The apparatus 200 includes: at least one processor 210 and at least one memory 212 including computer program code. The at least one memory 212 and the computer program code are configured to, with the at least one processor 210, cause the apparatus 200 at least to perform the following:

a) obtain electroencephalographic (EEG) data relating to a plurality of subjects diagnosed with ADHD (denoted using reference numeral 202);
b) extract, for each of the plurality of subjects, at least one feature from the EEG data relating to that subject (by an extractor 204);
c) formulating a prediction model (denoted using reference numeral 206) by performing regression analysis to map the extracted features against one or more markers for each of the plurality of subjects; and
d) determining that the prediction model provides an ADHD assessment (denoted using reference numeral 208) if one or more of the markers are indicators of a clinical measure of interest.

The EEG data may be from EEG signals measured by an EEG device 214, such as newly acquired data from an ADHD treatment session of the plurality of subjects diagnosed with ADHD. In addition, input for the regression analysis may include clinical data relating to the plurality of subjects, the clinical data being stored in the clinical data registration database 202b. The clinical data may be clinical ratings based on EEG data recorded in previous ADHD treatment sessions or the previous medical history of the plurality of subjects.

The at least one feature extracted by the extractor 204 may be a discriminative rhythmic feature. The discriminative rhythmic feature may be extracted using a complex-valued spatial-spectral filtering technique.

In one embodiment, the complex-valued spatial-spectral filtering technique may use an adapted form of Rayleigh coefficient as an objective function for system optimization.

The complex-valued spatial-spectral filtering technique may be a linear-phase technique. The linear-phase complex-valued spatial-spectral filtering technique may include transforming a segment of the EEG data into the frequency domain; computing a spatial energy feature at each of a number of given frequencies using a linear-phase complex-valued spatial-spectral filer and spectral coefficients of the EEG data segment; and summing computed spatial energy features over a range of frequency points of interest to obtain a spatial-spectral power feature.

The spatial energy feature may be computed according to the following expressions:

y_{f}=w_{f}x_{f}w_{f}

and

y_{f}=w_{f}x_{f}w_{f}

where y_{f} is the spatial energy feature at a given frequency f; w_{f} is the linear-phase complex-valued spatial-spectral filter; x_{f} are spectral coefficients of the EEG data segment; and T denotes a matrix transposition.

More detail on the Rhythmic feature extraction via linear-phase complex-valued filtering is provided below.

In one embodiment, a given EEG segment is transformed into frequency domain. At a frequency f, compute spatial energy feature by

y_{f}=w_{f}x_{f}w_{f} [1]

and

w_{f}=[w_{fl},K,w_{fn}]^{T} [2]

where w_{f} is the spatial filter, x_{f} the spectral coefficients of EEG. x_{f} has linear phase

w_{fi}=w_{f0}e^{jθ}^{ᵢ}^{f} [3]

and

w_{f}=w_{f}(w₀,θ) [4]

Adapt Rayleigh coefficient to be the objective function for learning

$$γ ( {w}_{0} , θ ) = \frac{{\sum }_{i} {\sum }_{f} {β}_{f} {w}_{f}^{T} ( {w}_{0} , θ ) {x}_{fi} {x}_{fi}^{T} {w}_{f} ( {w}_{0} , θ )}{{\sum }_{{i}^{′}} {\sum }_{f} {β}_{f} {w}_{f}^{T} ( {w}_{0} , θ ) {x}_{{fi}^{′}} {x}_{{fi}^{′}}^{T} {w}_{f} ( {w}_{0} , θ )} \qquad [ 5 ]$$

Numerical optimization may be used to solve the maximization of the function. For larger number of parameters, the use of real-value solutions (i.e. constraint satisfaction problem, CSP) may be performed as the initial step. The responsive frequency band may be selected by filter banks and mutual information-based criterion.

In another embodiment, the discriminative rhythmic feature may be extracted using a filtering technique based on a Bayes theorem. A feature extraction method using complex-valued filtering technique with a Bayes theorem is described below.

To obtain optimal rhythmic features, complex cross-spectrum in EEG can be processed in the frequency domain. Consider an EEG segment frequency representation by a matrix Xε [symbol] ⁿ^{_{c}}^{×n}^{_{f}}, with n_{c} number of channels.

$$X = [ \begin{matrix}{x}_{11} & … & {x}_{1 {n}_{f}} \\ ⋮ & ⋱ & ⋮ \\ {x}_{{n}_{c} 1} & … & {x}_{{n}_{c} {n}_{f}}\end{matrix} ] \qquad [ 6 ]$$

where xᵢⱼ denotes the discrete Fourier transform of the i-th channel at frequency

$${ω}_{j} = \frac{j - 1}{2 {n}_{f}} {F}_{s}$$

with Fs as the sampling frequency.

At each frequency f, there can be a particular spatial filter

w_{f}=[w₁(f), . . . ,wₙ_{_{c}}(f)] with wⱼ(f)=ŵⱼe^{iθ}^{ⱼ}^{(f)} [7]

Here ŵⱼ represents the magnitude of the complex-valued coefficient j, while θⱼ the phase. Particularly, a linear phase system is considered for the spatial filtering where the phases can be expressed by

θⱼ(f)=θⱼf [8]

The linear phase system ensures that all frequency components have equal delay times. This can be important for preserving phase synchronization patterns between channels across the frequency components. Another benefit is the number of free parameters reduced by a factor of n_{f}, and this can ease the optimization computation.

Consider w_{f} as a function:

w_{f}=u(f,\vec{θ},{circumflex over (\vec{w}) [9]

where \vec{θ} and {circumflex over (\vec{w} are the array of θⱼ in Eq. [8] and ŵⱼ in Eq. [7].

A DRF is the combination of the DRFs over all the frequency components in a selected window fₛₑₗ.

$$y = {\sum }_{f ∈ {f}_{sel}}^{ } {w}_{f}^{T} {R}_{f} {w}_{f} \qquad [ 10 ]$$

where Rf is the covariance matrix of EEG at frequency f. Consider Rayleigh coefficient for the objective function for learning. Rayleigh coefficient relates CSSF to contrast of rhythmic powers by describing the ratio between the energy of the transformed EEG class 1 (ω₁) and that of class 2 (ω₂), by a spatial filter w.

$$γ = \frac{{w}^{T} {R}_{1} w}{{w}^{T} {R}_{2} w} \qquad [ 11 ]$$

with R₁ and R₂ the covariance matrices for the two classes, respectively. Let ω₁ be the EEG class of interest, and ω₂ the other EEG class (idle state or other unrelated EEG class to contrast). It can be seen that minimizing Rayleigh coefficient is equivalent to enhancing the rhythmic attenuation feature in ω₁ in contrast to ω₂. Replace with ω₁ and ω₂ while maximizing the coefficient. Given a set of training samples, the empirical Rayleigh coefficient is given by

$$( 6 )$$

$$\hat{γ} = \frac{{\sum }_{j = 1}^{{n}_{o}} \text{?} {( u ( f , \vec{ϑ} , \vec{\hat{w}} ) )}^{T} {R}_{f 0 j} u ( f , \vec{ϑ} , \vec{\hat{w}} )}{{\sum }_{j = 1}^{{n}_{1}} \text{?} {( u ( f , \vec{ϑ} , \vec{\hat{w}} ) )}^{T} {R}_{f 1 j} u ( f , \vec{ϑ} , \vec{\hat{w}} )} \\ \text{?} \text{indicates text missing or illegible when filed} \qquad [ 12 ]$$

where R_{f0j} or R_{f1j} is the complex-valued covariance matrix of j-th EEG segment sample at frequency f in class ω₀ or class ω₁; and the objective of learning is to minimize the empirical Rayleigh coefficient, using readily available optimization tools for example, optimization tool found in MATLAB for the resolution to Eq. [13].

$$\vec{θ} , \vec{\hat{w}} = {arg min}_{\vec{θ} , \vec{\hat{w}}} \hat{γ} \qquad [ 13 ]$$

To find subject-specific rhythm for the most representative features, filter-bank technique in the spectral domain may be employed. Specifically, examining an array of frequency bands that cover the full range of EEG related to attention, and performing the above learning using each frequency band for Eq. [6]. This is followed by selecting the best frequency band for a particular task, e.g. for classification, the largest mutual information between features and class labels is selected (e.g. attention and non-attention), or for other analysis the largest inter-class contrast of features between two classes can be selected.

At this point, it is decided whether the use of complex-valued covariance matrices (including phase information) and complex-valued spatial filters lead to higher inter-class contrast in mean value (i.e. Rayleigh coefficient, which also determines classification accuracy) than real-value solutions.

The theorem described in Eqs. [19] to [29] under the heading “Example” provided below establish that real-value solutions are optimal only under a special condition, while generally complex-valued solutions should be preferred.

The discriminative rhythmic features obtained are used to compute three scores, namely: a BCI-ADHD severity measurement (BASM); a BCI-ADHD response prediction (BARP) and a ADHD severity change (ASCP).

To recap, the BASM provides an ADHD severity assessment score at the time of recording EEG data. The BARP provides an ADHD predictor score that predicts a change in the ADHD severity assessment score from a particular point of time to a point of time in the future, so as to provide an assessment of an ADHD treatment. The ASCP provides an ADHD response score that shows a change in the ADHD severity assessment score in between two EEG recording times. A different regression analysis may be used to obtain each of the ADHD severity assessment score, the ADHD response score and the ADHD change score.

For computing BASM, the following regression function ƒ_{basm}(y), was constructed, where ƒ_{basm} is constructed to minimize the empirical error to a given score θ which is the target ADHD severity rating score on inattentiveness.

$$min \{ {e}_{basm} = {\sum }_{i = 1}^{N} {( {f}_{basm} ( {\overline{y}}_{i} ) - {θ}_{i} )}^{2} \} \qquad [ 14 ]$$

where i denotes one of the N subjects; and y̅ᵢ is the mean feature vector of the subject.

For computing BARP, the following regression function was constructed:

ƒ_{barp}(y,t₀,t₁) [15]

where t₀ is the present time point (EEG recording for y) and t₁ is the time for prediction of ADHD condition. And ƒ_{BARP} is constructed to minimize the empirical error to empirical change of a given score θ in the time frame.

$$min \{ {e}_{barp} = {\sum }_{i = 1}^{N} {( {f}_{barp} ( {\overline{y}}_{i} ) - ( {θ}_{i} ( {t}_{1} ) - θ i ( {t}_{0} ) ) )}^{2} \} \qquad [ 16 ]$$

For computing ASCP, we construct the following regression function:

$${f}_{ascp} ( {y}_{0} , {y}_{1} , {t}_{0} , {t}_{1} ) \qquad [ 17 ]$$

where t₀ and t₁ are two time points and y0 and y1 are the then feature vector. And fascp is constructed to minimize the empirical error to empirical change of a given score θ in the time frame.

$$min \{ {e}_{barp} = {\sum }_{i = 1}^{N} {( {f}_{barp} ( {\overline{y}}_{i} ( {t}_{0} ) , {\overline{y}}_{i} ( {t}_{1} ) , {t}_{0} , {t}_{1} ) - ( {θ}_{i} ( {t}_{1} ) - {θ}_{i} ( {t}_{0} ) ) )}^{2} \} \qquad [ 18 ]$$

The above regressions were validated to evaluate their effectiveness, such as against EEG data obtained from a brain-computer-interface device.

The evaluation is based on a prospective single arm, open-label study. Participants in this validation, underwent 24 individual BCI intervention sessions over 2 months (3 sessions per week), followed by 3 months of once-monthly booster sessions, according to a manual protocol. Each session lasted 30 minutes. They were last assessed at 6 month after the first visit. Observers (e.g. parents of) of the participants completed the ARS-IV (ADHD Rating Scale-IV) at baseline, 2, 5 and 6 months. A parametric computation model, which mapped EEG signals into a numerical value (the BCI score) that indicates the subject's attention level, was built from the EEG data collected from a calibration session. During calibration, subjects completed a colour Stroop task interspersed with resting conditions. These tasks represent attention and non-attention states of the subject. The parametric computation model included a filter bank array and a linear regression. The filter banks decompose EEG into a continuous array of frequency sub-bands that cover the range from 4 Hz to 36 Hz. Machine learning techniques were used to derive a linear regression mapping from the band powers into the BCI score. This model was built in a subject-dependent manner to capture specific EEG characteristics for each individual.

FIG. 3 plots the correlation between BASM (Week 0) and the change in ADHD rating scores from Week 0 to Week 20 for ADHD inattention (denoted using reference numeral 302) and hyperactivity (denoted using reference numeral 304) symptoms, and a combination of both (denoted using reference numeral 306). The plots show that a linear form of ƒ_{barp} will be able to achieve significant correlation with the changes. This linear prediction model, formulated from EEG data and clinically obtained ADHD scores may be applied for ADHD assessment of a new subject.

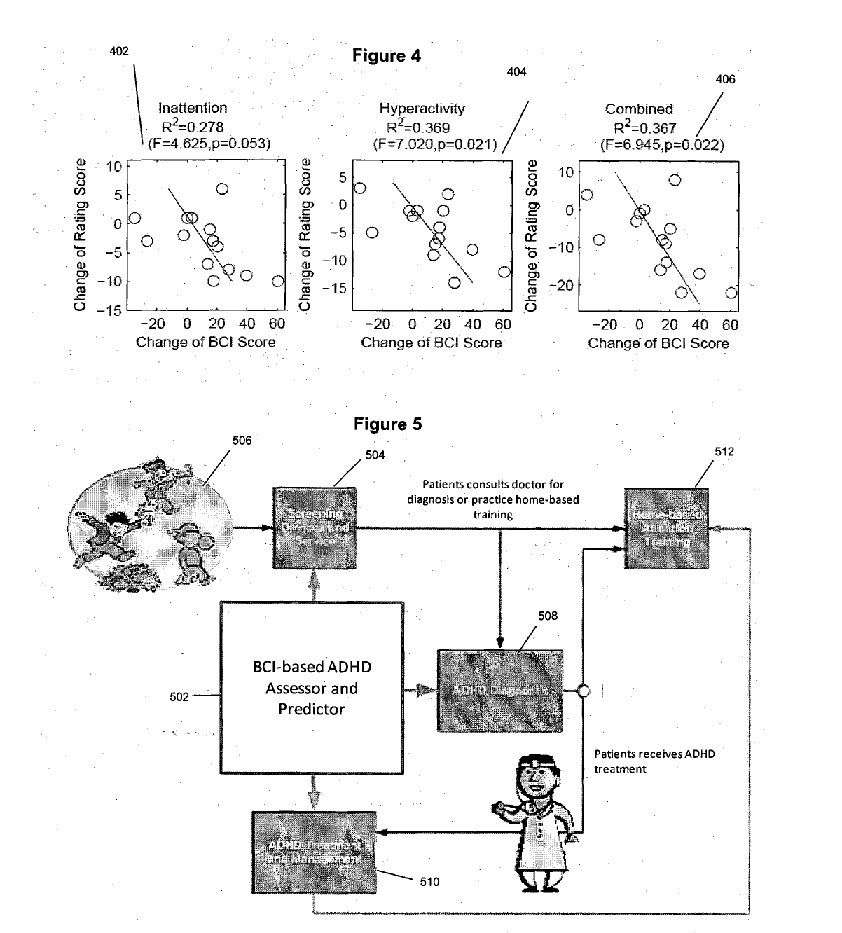

FIG. 4 plots the correlation between the change of BASM and the change in ADHD rating scores from Week 0 to Week 20 for ADHD inattention (denoted using reference numeral 402) and hyperactivity (denoted using reference numeral 404) symptoms, and a combination of both (denoted using reference numeral 406). Here the BASM is the same BCI score as above. The plots show that a linear form of f_{ascp} will be able to achieve significant correlation with the changes.

From the above, a BCI-based tool for objective ADHD clinical assessment and treatment responses using EEG and/or clinical data is provided. Various embodiments execute machine learning of both EEG data and clinical data, wherein EEG data may be based on Stroop test vs. resting protocol from a large pool of subjects; and the Clinical data is based on specific clinical ratings or their derivatives such as DSM-IV from a large pool of subjects. A feature extraction method, for example using complex-valued filtering technique, may be used to obtain features from this EEG data and clinical data and regression performed for associating the extracted features with EEG severity.

Various embodiments provide an apparatus that computes ADHD severity measure which represents the severity of ADHD at the time of EEG recording and is also associated with a given specific clinical measure of interest. A BCI-ADHD response predictor is computed which represents the estimated improvement of ADHD condition in terms of BASM from the time of EEG recording to a specified time. An ADHD Severity Change Predictor is computed which represents the estimated improvement of ADHD condition in terms of BASM in between two EEG recoding times.

The apparatus enables accurate clinical assessment using EEG only or with EEG plus clinical data and facilitates planning and tracking of progress of BCI-based treatment.

The apparatus provides a BCI-based methodology for ADHD clinical assessment and treatment predictions that acquires and stores subject-specific EEG data and clinical data; performs machine learning and extraction of discriminative rhythmic features (DRFs) from EEG using special complex-value spatial-spectral filters.

The apparatus learns and applies a regression function that maps EEG features and/or clinical data into a score named BCI ADHD Severity Measure (BASM), which represents the severity of ADHD at the time of EEG recording and is also associated with a given specific clinical measure of interest.

The apparatus learns and applies a regression function that maps EEG features and/or clinical data into a score named BCI-ADHD Response Predictor (BARP), which represents the estimated improvement of ADHD condition in terms of ASM from the time of EEG recording to a specified time.

The apparatus learns and applies a regression function that maps two sets of EEG features recorded at different times and/or clinical data into a score named ADHD Severity Change Predictor (ASCP), which represents the estimated improvement of ADHD condition in terms of ASM in between the two EEG recoding times.

FIG. 5 illustrates an exemplary application for the above embodiments. In an embodiment, BASM is provided by a BCI-based ADHD assessor and predictor 502 to screening devices and services 504. These screening devices and services 504 acquire EEG data from subjects 506 with ADHD. BASM may also be provided by the BCI-based ADHD assessor and predictor 502 to an ADHD diagnostic tool 508. BARP and ASCP may be provided by the BCI-based ADHD assessor and predictor to an ADHD treatment and management analyser 510. The analysis performed by the screening devices and services 504, the ADHD diagnostic tool 508 and the ADHD treatment and management analyser 510 may be used for further analysis by home-based attention training equipment 512.

A third embodiment also provides a computer readable medium for assessing the treatment of attention-deficit/hyperactivity disorder (ADHD) in a subject. The computer readable medium has stored thereon computer program code which when executed by a computer causes a computer to perform at least the following: obtaining electroencephalographic (EEG) data relating to a plurality of subjects diagnosed with ADHD; extracting, for each of the plurality of subjects, at least one feature from the EEG data relating to that subject; formulating a prediction model by performing regression analysis to map the extracted features against one or more markers for each of the plurality of subjects; and determining that the prediction model provides an ADHD assessment if one or more of the markers are indicators of a clinical measure of interest.

### Experiments and Discussion

A study was conducted having the following inclusion and exclusion criteria:

Inclusion criteria: A subject was eligible for inclusion in the study only if all the following criteria applied at pre-study screening:
Subject's age was within the age range of 6-12 years old;
Subject had never received treatment with stimulant medication

or Atomoxetine;
The subject should satisfy the following criteria for the

diagnosis of ADHD:
i) DSM-IV-TR criteria for ADHD, either the combined or inattentive subtype, based on clinical assessment
ii) Diagnostic Interview Schedule for Children (DISC), as
completed by the parents;
Written Informed Consent from parent and Assent Form from

child were both obtained;
Subject and the parent/guardian were willing to comply with study procedures and were able to return to the clinic for scheduled visits.

Exclusion criteria: A subject was not eligible for inclusion in the study if any of the following criteria applied at pre-study screening:
Present or history of medical treatment with stimulant medication and Atomoxetine;
Co-morbid severe psychiatric condition or known sensorineural deficit e.g. complete blindness or deafness (such that they could not play computer games);
History of epileptic seizures;
Known mental retardation (i.e. IQ 70 and below);
Predominantly Hyperactive/impulsive subtype of ADHD (i.e. no predominant inattentive symptoms).

20 participants were recruited for the study, including 16 males and 4 females. The mean age was 7.80 (SD=1.40, range 6-11). There were 17 Chinese, 2 Eurasians and one Malay. Fourteen children were diagnosed to have the combined subtype of ADHD based on C-DISC, and the other 6 had the inattentive subtype of ADHD.

The BCI-Based Attention Training Game System

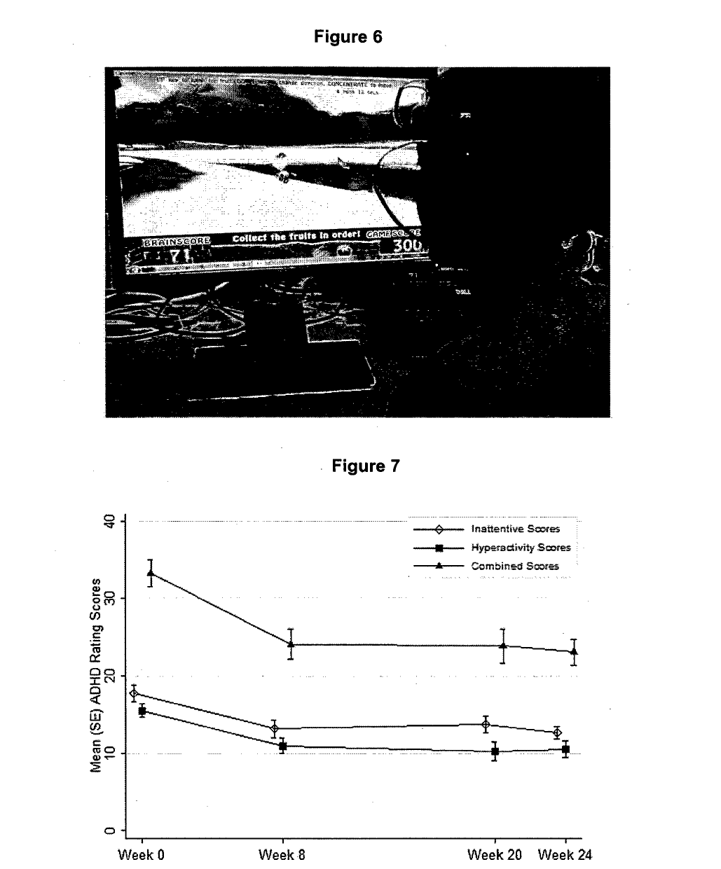

The BCI system consisted of a headband with mounted dry EEG sensors (manufactured by Neurosky, Inc) that transmitted EEG readings to the computer through Bluetooth-enabled protocol. The headband was worn around the forehead, with a grounding reference electrode clipped to the earlobe (see FIG. 6).

Two dry EEG electrode sensors positioned to detect the EEG pattern from the frontal sites FP1 and FP2 were mounted on a headband. The advanced signal processing techniques in the brain-computer interface can pick up useful information about attentional activities from the frontal EEG recorded at sites Fp1 and Fp2.

The possible effects of noise or artifacts such as extraocular activity on the EEG were considered and reduced in the BCI system. Since the noise and artifacts were generally uncorrelated with the attentiveness and in-attentiveness conditions, they were filtered out in our machine learning algorithm that extracted only discriminative features from EEG between the two conditions. To further reduce the electrooculography artifacts, a virtual EEG channel was added, which was the differential potential between Fp1 and Fp2. As a result, the system was not affected by normal eye movements.

Calibration:

Prior to playing the video game (CogoLand), which was the main training activity, each participant underwent individual calibration using a colour Stroop task on the BCI-based attention training game system. During calibration, the participant performed the colour Stroop task to develop an individualized EEG profile of the optimal attentive state. The colour Stroop task required one to use the mouse to click on the name of the colour in which a word was spelt, and not the colour that the word spelt.

The BCI-based attention training game system analyzed the critical EEG parameters during the correct attempts, compared to that when the participant was relaxing, to derive an individualized EEG pattern representing the participant's most attentive state

Playing the Game

A computerized 3D graphic game, CogoLand was developed specifically as the training game. In CogoLand, the participant controlled an avatar via the signals detected by the EEG electrodes. This was computed into a BCI ADHD Severity Measure, or BASM (see next section for details of its computation). The BASM was then transformed to a score ranging from 0 (minimum attention) to 100 (maximum attention), which was reflected on the computer screen. The participant would hence need to ‘concentrate’ in order to move the avatar, which would move at a speed proportional to the participant's attention level as measured by the BCI-based attention training game. The ‘higher’ the concentration level of the participant was, the higher would be the speed of the avatar's movement. There were three difficulty levels in CogoLand. The main goal of the first level was to make the avatar run around an island in the shortest time possible. The next two levels had an additional component where the child needed to collect a series of fruits floating in the air as the avatar navigated through a pre-determined route in a colorful town. The child would use a specific key on the keyboard to make the avatar jump to collect the fruits which would appear along the journey. The child was asked to collect as many fruits as possible within a given timeframe, after which the number of fruits collected was entered into a personal logbook. At the third level the child had to collect the fruits in an order presented on the screen. A short break was allowed between attempts. For each training session, the individual would complete 30 minutes of training, including the breaks.

BCI ADHD Severity Measure (BASM).

All raw EEG data obtained during calibration with the Stroop task was analyzed by the BCI system. It was screened to detect any abnormality in the EEG recordings, such as disconnected electrodes and saturated digital samples. Any abnormal EEG readings, including the readings within two seconds from the occurrence of the abnormality were excluded from analysis. The system then extracted discriminative rhythmic power features from the screened EEG using spatial-spectral filtering.

The system examined an array of 8 frequency bands, continuously covering from 4 Hz to 30 Hz. This arrangement not only covered traditional EEG bands from theta to beta, but also had a finer grid of frequency bands. Band powers were computed using the following procedure. First, the EEG data was segmented into a continuous sequence of 2-second long time blocks; in each block, power spectrum was computed using a 256-point Fast-Fourier-Transform technique; a specific band power was calculated as the sum of the spectrum powers at all the discrete frequencies in the band; and the specific band power was the average value of the band power over the time blocks. The band power was calculated for each EEG channel separately, in addition to the differential potential between the two channels.

The BCI system then selected the band power features for maximizing the separation between attentive and inattentive states according to the information theory. A regression function would be applied by the BCI system to transform the selected features into a BASM score, which represented the severity of the inattentive symptoms of ADHD at the time of EEG recording. The BASM score was inversely proportional to the severity of the inattentive symptoms and the lower the BASM score, the more inattentive the individual was.

Treatment Program

This BCI-based attention training game system was used as an intervention program (the BCI-based attention training program), which comprised of an intensive phase with 3 training sessions weekly for 8 weeks, followed by a maintenance, phase with once-monthly booster training sessions for 3 consecutive months. At the end of every alternate training session starting from the second session, each participant would complete 2 short English and Mathematic worksheets consisting of multiple choice type questions on the computer. These worksheets were appropriate to their educational level and took approximately 10 minutes to complete. Each participant was instructed to concentrate like when they were playing the games, and their EEG was monitored during this period. Treatment was administered by 3 therapists trained to fit the headband and administer the BCI based training program. All the therapists had obtained at least a graduate degree in psychology, and they administered treatment according to a standardized treatment protocol. Calibration was done at weeks 0, 4 and 8.

Study Outcome Measures

At baseline, parents completed the 18-question ADHD Rating Scale, 4th edition (ADHD-RS). The ADHD-RS was based on the DSM-IV criteria for ADHD and consisted of nine inattentive and nine hyperactive-impulsive symptoms, with a four-point scale (0=never [less than once a week], 1=sometimes [several times a week], 2=often [once a day], and 3=very often [several times a day]). Three measures were taken from the ADHD-RS: inattentive (IA) score (0-27), hyperactive-impulsive (HI) score (0-27) and combined (COM) score (0-54). The ADHD-RS was completed again at the end of weeks 4, 8, 20 (post-boosters), and 24. The primary outcome measures were the changes in ADHD-RS at weeks 8 and 20, compared to the baseline, to examine the efficacy of the intensive training and booster training sessions respectively. Additionally EEG information was collected during each training session to examine for any significant EEG change.

Results

Study Completion and Dropout

There were 17 (85%) subjects who completed the entire study. One boy dropped out before 4 weeks, as the parent felt there was no improvement in the child's behaviour. Two other boys dropped out between 4 and 8 weeks due to difficulty adhering to the treatment schedule.

ADHD Rating Scale-IV Results

Intention-To-Treat (ITT) analyses was conducted on the results and excluded the subject who dropped out before week 4 as there was no follow up data at all. We carried forward the last available observation (behavioural rating) where appropriate.

Multiple imputations using Markov chain Monte Carlo and a per protocol analysis including only subjects who completed the study were also conducted and the results did not show much difference from that based on the last observation carried forward method.

TABLE 1
ADHD rating scale IV (ARS-IV) inattentive (IA), hyperactive-impulsive
(HI) and combined symptoms (COM) total raw scores as rated by parents

| | | Inattentive | Hyperactive- | |
|---|---|---|---|---|
| | | (IA) | Impulsive | Combined (COM) |
| Week 0 | Sample Size | 19 | 19 | 19 |
| | Mean (SD) | 17.7 (5.0) | 15.6 (3.9) | 33.4 (7.8) |
| Week 8 | Sample Size | 19 | 19 | 19 |
| | Mean (SD) | 13.1 (5.0) | 10.9 (4.4) | 24.1 (8.5) |
| Week 20 | Sample Size | 17 | 17 | 17 |
| | Mean (SD) | 13.6 (4.5) | 10.2 (5.1) | 23.8 (8.9) |
| Week 24 | Sample Size | 17 | 17 | 17 |
| | Mean (SD) | 12.6 (3.4) | 10.5 (4.3) | 23.1 (6.9) |

Table 1 summarizes parent-rated ADHD-RS scores at various study visits for the participants included in the analysis. No deviation from normal distribution was found for the ADHD-RS scores using both normality tests and graphic methods. Changes of these scores at week 8 and 20 from baseline were assessed by the paired t-test. Similarly, mean changes in these scores at week 20 from week 8 and changes at week 24 from baseline were analyzed to determine any booster effect and long-term effect, respectively. At week 8, the mean (SD) change compared to week 0 for inattentive (IA) symptoms was 24.6 (5.9) and the median (range) change was 23.0 (217.0, 4.0). It was shown that this median change was statistically significant (p=0.003). Similarly, the mean changes in parent-rated hyperactive impulsive (HI: mean change=24.7 (5.6)) and combined (COM: mean change=29.3 (11.0)) symptoms were statistically significant (p=0.002 for both HI and COM).

There was no statistically significant change in parental observation of inattentive and hyperactive-impulsive symptoms on the ADHD-RS at 20 weeks compared to 8 weeks, or at 24 weeks compared to 20 weeks. When examining the ratings at 24 weeks, compared to the baseline score, there was significant improvement in parent-rated inattentive and hyperactive-impulsive symptoms (mean changes=25.0 (5.8) and 25.7 (5.1) respectively and p≦0.01 for both IA and HI). These results appear to suggest that monthly booster training for 3 consecutive months after an intensive 8-week training did not significantly improve inattentive or hyperactive-impulsive symptoms further. The behavioural benefits from the intensive training at 8 weeks were sustained at 24 weeks. The child's age and gender did not have any statistically significant effect on ADHD-RS scores in this study.

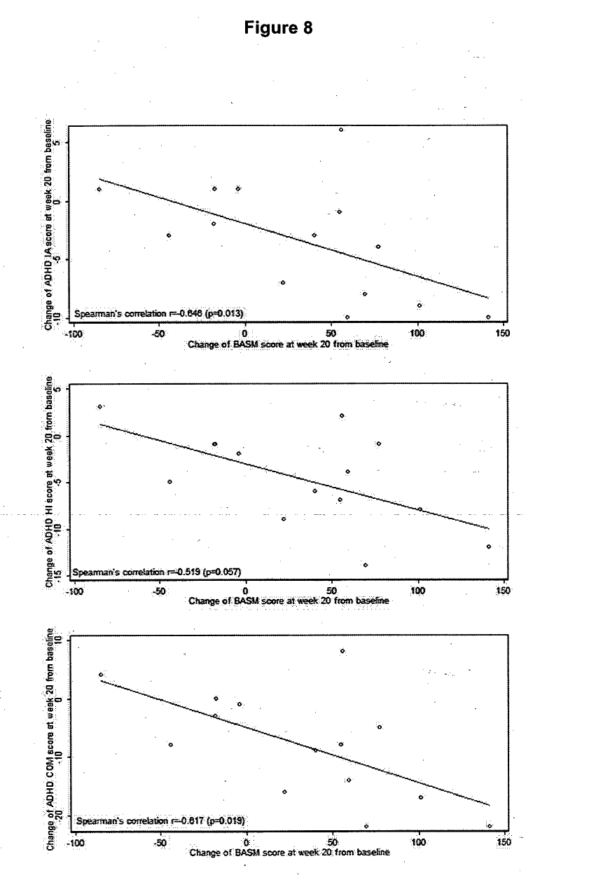

FIG. 7 is a graphical representation of the change in mean and standard error of the ADHD-RS scores as rated by parents over the 24 weeks duration of the study.

EEG Results

EEG data from the calibration/re-calibration sessions was used (at week 0 and 20) and examined the BASM scores. The 3 participants who dropped out before week 8 and 3 other patients who had missing EEG data at Week 20 were excluded. Thus, a total of 14 participants from the original 20 recruited were analysed. When comparing the BASM scores at Week 0 and at Week 20, there was an increase in the mean score (standard deviation) from 60.9 (81.0) to 96.9 (64.7), although paired t-test showed that the change was not statistically significant (mean change=32.5 (60.8), p=0.067).

Predictors of Clinical Outcome

Linear regressions showed that baseline IA, HI and COM scores statistically significantly predicted their respective changes of ADHD rating scale scores from week 0 to 8 (b (SE)=20.7 (0.2), 20.9 (0.3) and 20.9 (0.3) respectively and p=0.013, 0.008 and 0.007 respectively). Thus, a higher score on the ADHD-RS at baseline predicts greater improvement at week 8. The possible correlations between the change in BASM scores and the changes in scores on ADHD rating scale from Week 0 to Week 20 were investigated. Correlation analyses performed in FIG. 8 showed that there were strong negative correlations between changes in these two scores (Spearman's correlation coefficients were 20.646, 20.519, and 20.617 for IA, HI and COM respectively). This was statistically significant for both IA (p=0.013) and COM (p=0.019), but not for HI (p=0.057). In other words, increasing BASM scores was associated generally with decreasing ADHD scores. Age, gender or ADHD subtype was not found to predict the ADHD-RS changes at weeks 8 or 20.

Discussion

The study evaluated a BCI-based attention training program, according to various embodiments, which included dry sensors and blue tooth technology in place of EEG leads with a game CogoLand, in the treatment of combined and inattentive subtypes of ADHD. The results showed that an 8-week intervention significantly improved inattentive symptoms of ADHD, based on a behavioural rating scale by parents. Among children with the combined subtype of ADHD, parents also reported a significant improvement in their hyperactive-impulsive symptoms on the ADHD Rating Scale. When these children received monthly training sessions subsequently, the behavioural improvements were sustained but did not further improve. Those with more severe symptoms were also the ones who showed greater improvement.

It was found that the change in BASM score correlated with the change in behavioural rating score by parents. This provided some evidence that the improvement with training, as reflected by the BASM score, might explain the improved ADHD symptoms reported by parents. The BASM score also appeared to be a good surrogate marker for observed inattentive behaviour.

The results show a refinement from previous work that looked at specific EEG bands. Neurophysiological studies have previously shown that children with ADHD exhibit specific patterns on the electroencephalogram (EEG) and this has been utilized clinically to diagnose, treat and even predict response to treatment with medication [14,15,16,17,18]. EEG studies of children with ADHD showed the majority to exhibit abnormal patterns of resting cortical activity including increased slow-wave activity (primarily theta waves), decreased fast-wave activity (primarily beta waves) and increased beta-theta ratio.

These findings are consistent with the inattentive symptoms exhibited in ADHD, as beta activity is associated with concentration or mental activity whereas theta activity is associated with drowsiness. During the performance of cognitive tasks, children with ADHD exhibit EEG changes similar differences compared to normal matched controls. Through childhood EEG, it was also possible to predict those at risk of having ADHD symptoms which persisted into adulthood.

BCI-based attention training game system can offer several advantages over current evidence-based treatment options offered in most clinical practices. It has less adverse events compared to medication. Unlike behavioural management or parent training, there is no need for regular clinic visits which can be inconvenient.

It will be appreciated by a person skilled in the art that numerous variations and/or modifications may be made to the present invention as shown in the embodiments without departing from a spirit or scope of the invention as broadly described. The embodiments are, therefore, to be considered in all respects to be illustrative and not restrictive.

### Example

Let R₁ and R₂ be the covariance matrices of two equal-prior-probability EEG classes ω₁ and ω₂, with the transformed feature y being a random variable from an exponential distribution. The optimal solution to minimization/maximization of Rayleigh coefficient, which is equivalent to maximization of inter-class contrast, is real value only if the following criterion is satisfied:

(Rᵣ₂⁻¹ Rᵣ₁) must share with (Rᵢ₂⁻¹ Rᵣ₁) the same eigen vector corresponding to the same minimal eigen value; where R₁=Rᵣ₁+iRᵢ₁ and R₂=Rᵣ₂+iRᵢ₂, with real-value matrices Rᵣ and Rᵢ being the real part and the imaginary part of a covariance matrix (R₁ or R₂).

Considering a complex-valued filter w can be written in real and imaginary parts:

w=wᵣ+iwᵢ [19]

From the definition of Rayleigh coefficient given in Eq. [11], the equation is rewritten as

$$ε = \frac{{( {w}_{r} + {iw}_{i} )}^{T} ( {R}_{r 1} + {iR}_{i 1} ) ( {w}_{r} + {iw}_{i} )}{{( {w}_{r} + {iw}_{i} )}^{T} ( {R}_{r 2} + {iR}_{i 2} ) ( {w}_{r} + {iw}_{i} )} \qquad [ 20 ]$$

Note that the operator T denotes conjugate transpose: (wᵣ+iwᵢ)^{T}=wᵣ^{T}−iwᵢ. Furthermore, Rᵢ has all 0 diagonal elements and Rᵢ=−Rᵢ. Thus, the above equation can be further developed into

$$ε = \frac{{w}_{r}^{T} {R}_{r 1} {w}_{r} + {w}_{i}^{T} {R}_{r 1} {w}_{i} - 2 {w}_{r}^{T} {R}_{i 1} {w}_{i}}{{w}_{r}^{T} {R}_{r 2} {w}_{r} + {w}_{i}^{T} {R}_{r 2} {w}_{i} - 2 {w}_{r}^{T} {R}_{i 2} {w}_{i}} \qquad [ 21 ]$$

Therefore, the partial derivative of the Rayleigh coefficient with respect to the real part and the imaginary part of the spatial filter w is

$$\begin{matrix}\frac{\partial ε}{\partial {w}_{r}} = - \text{?} [ ( 2 {R}_{r 1} {w}_{r} + 2 {w}_{i}^{T} {R}_{i 1} ) ( {w}_{r}^{T} {R}_{r 2} {w}_{r} + {w}_{i}^{T} {R}_{r 2} {w}_{i} - 2 {w}_{r}^{T} {R}_{i 2} {w}_{i} ) - ( 2 {R}_{r 2} {w}_{r} + 2 {w}_{i}^{T} {R}_{i 2} ) ( {w}_{r}^{T} {R}_{r 1} {w}_{r} + {w}_{i}^{T} {R}_{r 1} {w}_{i} - 2 {w}_{r}^{T} {R}_{i 1} {w}_{i} ] , or & [ 22 ] \\ \frac{\partial ε}{\partial {w}_{i}} = - \text{?} [ ( 2 {R}_{r 1} {w}_{i} + 2 {w}_{r}^{T} {R}_{i 1} ) ( {w}_{r}^{T} {R}_{r 2} {w}_{r} + {w}_{i}^{T} {R}_{r 2} {w}_{i} - 2 {w}_{r}^{T} {R}_{i 2} {w}_{i} ) - ( 2 {R}_{r 2} {w}_{r} - 2 {w}_{i}^{T} {R}_{i 2} ) ( {w}_{r}^{T} {R}_{r 1} {w}_{r} + {w}_{i}^{T} {R}_{r 1} {w}_{i} - 2 {w}_{r}^{T} {R}_{i 1} {w}_{i} ] , \\ \text{?} \text{indicates text missing or illegible when filed} & [ 23 ]\end{matrix}$$

with K the square of the denominator in Eq. [21]. The optimum solution wₒₚₜ to minimization/maximization of the Rayleigh coefficient ε must satisfy both

$$\frac{\partial ε}{\partial {w}_{i}} = 0 and \frac{\partial ε}{\partial {w}_{r}} = 0.$$

Then condition for wₒₚₜ to be a real-value vector is that

$$\begin{matrix}\frac{\partial ε}{\partial {w}_{r}} {\vert}_{{w}_{i} = 0} = 0 & [ 24 ] \\ \frac{\partial ε}{\partial {w}_{i}} {\vert}_{{w}_{i} = 0} = 0 & [ 25 ]\end{matrix}$$

From Eq. [24].

$${R}_{r 1} {w}_{r} = ( \frac{{w}_{r}^{T} {R}_{r 1} {w}_{r}}{{w}_{r}^{T} {R}_{r 2} {w}_{r}} ) {R}_{r 2} {w}_{r} \qquad [ 26 ]$$

The expression in the bracket is exactly the Rayleigh coefficient ε when wᵢ=0. This equation naturally leads to the generalised eigen value solution

(Rᵣ₂⁻¹Rᵣ₁)wᵣ=ε [27]

This equation is the solution to CSP. By introducing Eq. [26] to Eq. [25],

$$\begin{matrix}{R}_{i 1} {w}_{r} = ( \frac{{w}_{r}^{T} {R}_{r 1} {w}_{r}}{{w}_{r}^{T} {R}_{r 2} {w}_{r}} ) {R}_{i 2} {w}_{r} and & [ 28 ] \\ ( {R}_{i 2}^{- 1} {R}_{i 1} ) {w}_{r} = ε & [ 29 ]\end{matrix}$$

Consider both Eq. 21 and Eq. 23. For the solution wᵣ to satisfy both conditions, (Rᵣ₂⁻¹Rᵣ₁) must share with (Rᵢ₂⁻¹Rᵣ₁) the same eigenvector corresponding to the same minimal eigen value. Thus, the theorem is proven.

1. A method for assessing the treatment of attention-deficit/hyperactivity disorder (ADHD) in a subject, the method comprising:
obtaining electroencephalographic (EEG) data relating to a plurality of subjects diagnosed with ADHD;
extracting, for each of the plurality of subjects, at least one feature from the EEG data relating to that subject;
formulating a prediction model by performing regression analysis to map the extracted features against one or more markers for each of the plurality of subjects; and
determining that the prediction model provides an ADHD assessment if one or more of the markers are indicators of a clinical measure of interest.

2. The method of claim 1, further comprising retrieving clinically obtained ADHD scores relating to the plurality of subjects; and using the clinically obtained ADHD scores as input to the regression analysis, to formulate the prediction model.

3. The method of claim 1, wherein the EEG data comprises historical EEG data relating to the plurality of subjects or is obtained from a brain-computer-interface device.

4. The method of claim 1, wherein the at least one feature is a discriminative rhythmic feature.

5. The method of claim 4, wherein the discriminative rhythmic feature is extracted using a complex-valued spatial-spectral filtering technique.

6. The method of claim 5, wherein the complex-valued spatial-spectral filtering technique uses an adapted form of Rayleigh coefficient as an objective function for system optimization.

7. The method of claim 5, wherein the complex-valued spatial-spectral filtering technique is a linear-phase technique.

8. The method of claim 7, wherein the linear-phase complex-valued spatial-spectral filtering technique comprises:
transforming a segment of the EEG data into the frequency domain;
computing a spatial energy feature at each of a number of given frequencies using a linear-phase complex-valued spatial-spectral filer and spectral coefficients of the EEG data segment; and
summing computed spatial energy features over a range of frequency points of interest to obtain a spatial-spectral power feature.

9. The method of claim 8, wherein the spatial energy feature is computed according to the following expressions:
y_{f}=w_{f}x_{f}y_{f}
and
y_{f}=w_{f}x_{f}w_{f}
where y_{f} is the spatial energy feature at a given frequency f; W_{f} is the linear-phase complex-valued spatial-spectral filter; X_{f} are spectral coefficients of the EEG data segment; and T denotes a matrix transposition.

10. The method of claim 1, wherein the prediction model that is formulated depends on the extracted features that are mapped for the formulated prediction model to provide a different ADHD assessment score.

11. The method of claim 1, wherein the prediction model used to obtain each of the ADHD assessment is different.

12. The method of claim 1, wherein the prediction model used to obtain a first of the ADHD assessment is based on the prediction model used to obtain a second of the ADHD assessment.

13. The method of claim 1, wherein the ADHD assessment is an ADHD severity score at an instance of obtaining the EEG data for one subject.

14. The method of claim 1, wherein the ADHD assessment is a change score between two time points for one subject.

15. The method of claim 12, wherein the two time points are two instances of obtaining the EEG data for one subject, to provide an ADHD response score.

16. The method of claim 12, wherein the two time points are at an instance of obtaining the EEG data for one subject and another instance where the EEG data has yet to be obtained, to provide an ADHD predictor score.

17. The method of claim 1, wherein the ADHD assessment comprises:
an ADHD severity score at an instance of obtaining the EEG data for one subject; and
a change score between two time points for one subject, wherein the two time points are
two instances of obtaining the EEG data for the one subject, to provide an ADHD response score; or
an instance of obtaining the EEG data for the one subject and another instance where the EEG data has yet to be obtained, to provide an ADHD predictor score,
wherein the prediction model used to obtain the ADHD severity score, the ADHD response score and the ADHD predictor score is different.

18. The method of claim 1, wherein the ADHD assessment comprises:
an ADHD severity score at an instance of obtaining the EEG data for one subject; and
a change score between two time points for one subject, wherein the two time points are
two instances of obtaining the EEG data for the one subject, to provide an ADHD response score; or
an instance of obtaining the EEG data for the one subject and another instance where the EEG data has yet to be obtained, to provide an ADHD predictor score,
wherein the prediction model used to obtain the ADHD response score and the ADHD predictor score is based on the prediction model used to obtain the ADHD severity score.

19. An apparatus for assessing the treatment of attention-deficit/hyperactivity disorder (ADHD) in a subject, the apparatus comprising:
at least one processor; and
at least one memory including computer program code, the at least one memory and the computer program code configured to, with the at least one processor, cause the apparatus at least to perform:
obtaining electroencephalographic (EEG) data relating to a plurality of subjects diagnosed with ADHD;
extracting, for each, of the plurality of subjects, at least one feature from the EEG data relating to that subject;
formulating a prediction model by performing regression analysis to map the extracted features against one or more markers for each of the plurality of subjects; and
determining that the prediction model provides an ADHD assessment if one or more of the markers are indicators of a clinical measure of interest.

20. A computer readable medium for assessing the treatment of attention-deficit/hyperactivity disorder (ADHD) in a subject, the computer readable medium having stored thereon computer program code which when executed by a computer causes the computer to perform at least the following:
obtaining electroencephalographic (EEG) data relating to a plurality of subjects diagnosed with ADHD;
extracting, for each of the plurality of subjects, at least one feature from the EEG data relating to that subject;
formulating a prediction model by performing regression analysis to map the extracted features against one or more markers for each of the plurality of subjects; and
determining that the prediction model provides an ADHD assessment if one or more of the markers are indicators of a clinical measure of interest.
